(19) United States
(12) Patent Application Publication
(10) Pub. No.: US 2016/0287432 A1
(43) Pub. Date: Oct. 6, 2016

(54) SERPENTINE HEAT EXCHANGE ASSEMBLY FOR REMOVABLE ENGAGEMENT WITH PATIENT HEAT EXCHANGE SYSTEM

(71) Applicant: Zoll Circulation, Inc., San Jose, CA (US)

(72) Inventors: Jeremy Thomas Dabrowiak, Santa Clara, CA (US); Mark Davey, North Aurora, IL (US)

(21) Appl. No.: 14/675,421

(22) Filed: Mar. 31, 2015

(51) Int. Cl. A61F 7/12; A61F 7/08; F28D 9/00

(52) U.S. Cl. CPC A61F 7/12; F28D 9/0062; A61F 7/08; A61F 2007/0054

(57) ABSTRACT

A heat exchange assembly can be removably engaged between two cold plates of a heat exchange system for exchanging heat with working fluid from an intravascular heat exchange catheter or an external heat exchange pad. The heat exchange assembly may have a serpentine, winding, tortuous, or sinuous configuration, or it may have one or more curves, turns and/or bends and/or can have a flattened transverse cross-section to facilitate heat exchange with the cold plates.

### TECHNICAL FIELD

The present application relates generally to heat exchange -assemblies for removable engagement with patient heat exchange systems.

### BACKGROUND

Patient temperature control systems have been introduced to prevent fever in patients in the neuro ICU due to suffering from sub-arachnoid hemorrhage or other neurologic malady such as stroke. Also, such systems have been used to induce mild or moderate hypothermia to improve the outcomes of patients suffering from such maladies as stroke, cardiac arrest, myocardial infarction, traumatic brain injury, and high intracranial pressure. Moreover, such systems have been used for warming purposes such as for burn patients and other patients who may suffer from deleterious or accidental hypothermia. Examples of intravascular heat exchange catheters are disclosed in U.S. Pat. Nos. 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, all of which are incorporated herein by reference.

External patient temperature control systems may be used. Such systems are disclosed in U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein, by reference.

### SUMMARY

Accordingly, one aspect of a heat exchange member includes a hollow body through which working fluid from an intravascular heat exchange catheter or an external heat exchange pad can flow. The body is configured for removable engagement between two plates of a heat exchange system for exchanging heat between the heat exchange system and the working fluid. The body includes a working fluid inlet, a working feud outlet, and one or more or a plurality of straight segments between the inlet and outlet. Adjacent plural straight segments are connected by a respective curved end segment and at least some of the end segments are corrugated or flexible. The hollow body may have a serpentine, winding, tortuous, or sinuous configuration or it may have one or more curves, turns and/or bends.

Without limitation, the heat exchange member can be made of various materials, including metal or thermally conductive thermo-plastic.

In some implementations, one or more of the straight segments may define respective non-circular transverse cross-sections. The non-circular transverse cross-sections can be oblong and if desired can have opposed flat sides.

The heat exchange member may also include the plates, and the plates can be configured for circulating refrigerant to and from a compressor, which may also be included in certain embodiments. Likewise, other embodiments may include, along with the body, the catheter and/or the pad.

In another embodiment, a heat exchange assembly is removably engageable between two heat exchange (HEX) plates of a heal exchange system for exchanging heat with working fluid from an intravascular heat exchange catheter or an external heat exchange pad. The heat exchange assembly has plurality of bends or turns and a flattened transverse cross-section to facilitate heat exchange with the HEX plates.

In another embodiment, a heat exchange system includes first and second heat exchange (HEX) plates, and a heal exchange tubing assembly defining one or more fluid flow pathway with one or more flat segments in the fluid flow pathway. The tubing assembly is configured for being received between the HEX plates to exchange heat therewith.

The details of the various embodiments and aspects described herein, both as to structure and operation, can best be understood in reference to the accompanying drawings, in which like reference numerals refer to like parts, and in which:

### BRIEF DESCRIPTION OF THE DRAWINGS

FIG. 1 is a schematic view of a non-limiting system in accordance with an embodiment of the present invention;

FIG. 2 is a schematic plan view of a first embodiment of the heat exchange member with a circular transverse cross-section, showing only three corrugated end “turn” segments for clarity of disclosure, it being understood that a greater number of turns in the beat exchange member configuration, may be employed;

FIG. 3 is a transverse cross-section as seen along the line 3-3 in FIG. 2;

FIG. 4 is a perspective view of one of the two heat exchange or cold plates between which the heat exchange member of FIG. 2 can be removably disposed;

FIG. 5 is an exploded perspective view illustrating the heat exchange member of FIG. 2 between two heat exchange or cold plates that can he moved together to clamp the heat exchange member between them, schematically showing the compressor;

FIG. 6 is a side view of the assembly of FIG. 5 in the clamped configuration;

FIG. 7 is a perspective view of an alternate embodiment of a heat exchange member, the straight segments of which are configured with a non-circular transverse cross-section;

FIG. 8 is a plan view of an embodiment of the heat exchange member of FIG. 7 with spacer combs engaged with the tubing to maintain spacing;

FIG. 9 is a transverse cross-section as seen along the line 9-9 in FIG. 7;

FIG. 10 is a side view of a portion of one of the heat exchange or cold plates that may be used with the heat exchange member of FIG. 7, illustrating the non-circular groove configuration to match the non-circular transverse cross-section of the straight segments shown in FIG. 7; and

FIG. 11 is a side view of the heat exchange member of FIG. 7 showing the spacer combs in an exploded relationship with the heat exchange member.

### DETAILED DESCRIPTION

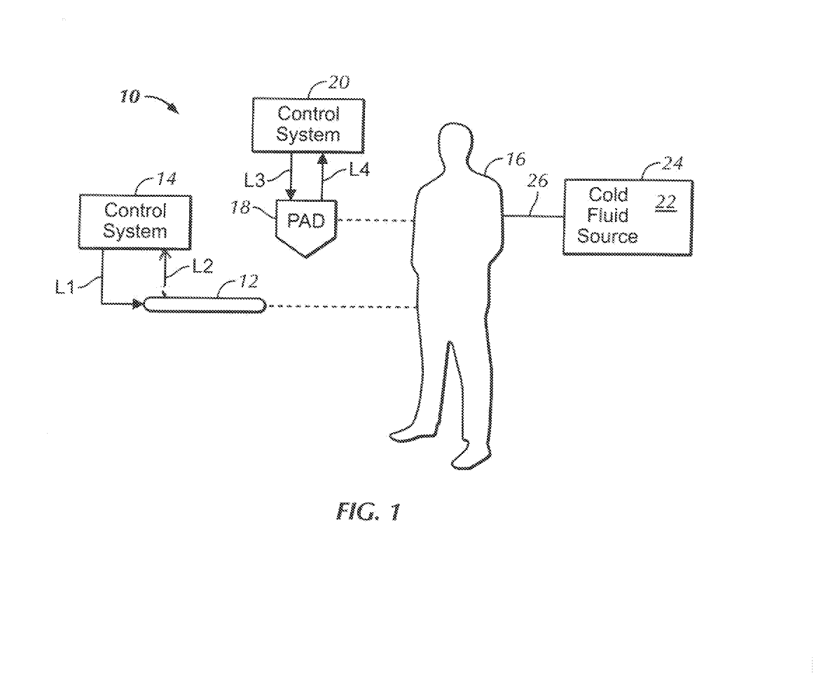

Referring initially to FIG. 1, in accordance with present principles, a system 10 may include an intravascular heat exchange catheter 12 controlled by a control system 14 to induce control patient temperature, e.g., to prevent the patient 16 from becoming febrile or to induce therapeutic hypothermia in the patient 16. In the catheter, working fluid or coolant, such as but not limited to saline, circulates (typically under the influence of a pump in the controller) in a closed loop from the control system 14, through a fluid supply line L1, through the catheter 12, and back to the system 14 through a fluid return line L2, such that no working fluid or coolant enters the body. While certain preferred catheters are disclosed below, it is to be understood that other catheters can be used in accordance with present principles, including, without limitation, any of the catheters disclosed above or in the following U.S. patents, all incorporated herein by reference; U.S. Pat. Nos. 5,486,208, 5,837,003, 6,110,168, 6,149,673, 6,149,676, 6,231,594, 6,264,679, 6,306,161, 6,235,048, 6,238,428, 6,245,095, 6,251,129, 6,251,130, 6,254,626, 6,261,312, 6,312,452, 6,325,818, 6,409,747, 6,368,304, 6,338,727, 6,299,599, 6,287,326, 6,126,684. The catheter 12 may be placed in the venous system, e.g., in the superior or inferior vena cava.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents incorporated herein by reference. The temperature of the pad 18 can be controlled by a pad control system 20 in accordance with principles set forth in the external pad patents to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia. The pad 18 may receive working fluid from the system 20 through a fluid supply line L3, and return working fluid to the system 20 through a fluid return line L4. Note that in some embodiments, the systems 14, 20 are established in a single assembly.

To cool the patient while awaiting engagement of the catheter 12 and/or pad 18 with the patient, cold fluid 22 in a cold fluid source 24 may be injected into the patient and in particular into the patient's venous system through a pathway 26. Without limitation, the pathway 26 may be an IV line, the source 24 maybe an IV bag, and the thud 22 maybe chilled saline, e.g., saline at the freezing point or slightly warmer. Or, the source may be a syringe, and the saline can be injected directly into the bloodstream of the patient.

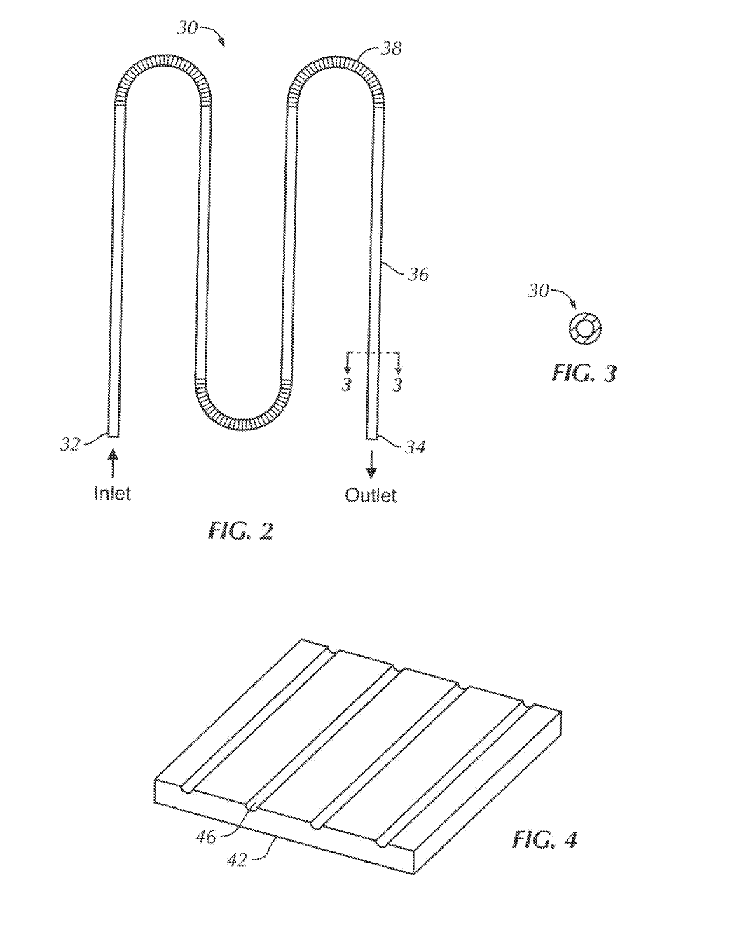

Now referring to a first embodiment of a heat exchange member assembly shown in FIGS. 2-6, a heat exchange member 30 has a hollow body or tubing as shown. The body may have a serpentine, winding, tortuous, or sinuous configuration, or it may have one or more curves, turns and/or bends. The simplified body shown in FIG. 2 has a fluid inlet 32 and a fluid outlet 34 to respectively receive and supply (typically through respective IV lines or other tubing with appropriate connectors such as Luer fittings) working fluid, to the catheter 12 and/or pad 18 in a closed fluid circuit.

Plural, generally co-parallel and co-planar straight segments 36 are located on the body between the inlet 32 and outlet 34. As shown, adjacent straight segments 36 are connected together by a respective curved end segment 38. One or more of or preferably all of the end segments 38 may be corrugated and/or flexible as shown to facilitate relatively tight bends in the end segments. It will be appreciated that in the example shown, the end segments 38 redirect working fluid about 180 degrees from one straight segment 36 to the immediately downstream straight segment 36. Thus, each end segment 36 may be semi-circular. The straight segments may or may not be corrugated or flexible. In other embodiments, the end segments may have a bent or otherwise angled configuration.

In the example of FIGS. 2-6, the straight segments 36 are cylindrical, and so have a circular transverse cross-section as shown in FIG. 3. To add or remove heat from the working fluid circulating through the heat exchange member 30, the heat exchange member 30 may he disposed between two heat exchange (HEX) plates 40, 42 (FIGS. 4-6), also referred to herein as cold plates without Loss of generality. It is to be understood that the HEX plates 40, 42 communicate with a compressor 44 to circulate refrigerant between the HEX plates and compressor to facilitate heat exchange between the working fluid and refrigerant via conduction through the HEX plates 40, 42 and heat exchange member 30. The HEX plates 40, 42 and compressor 44 may fee part of a control system shown in FIG. 1. In certain embodiments, refrigerant may be circulated, e.g., through fluid channels in the HEX plates, such that the HEX plates may maintain a temperature of about zero degrees Celsius.

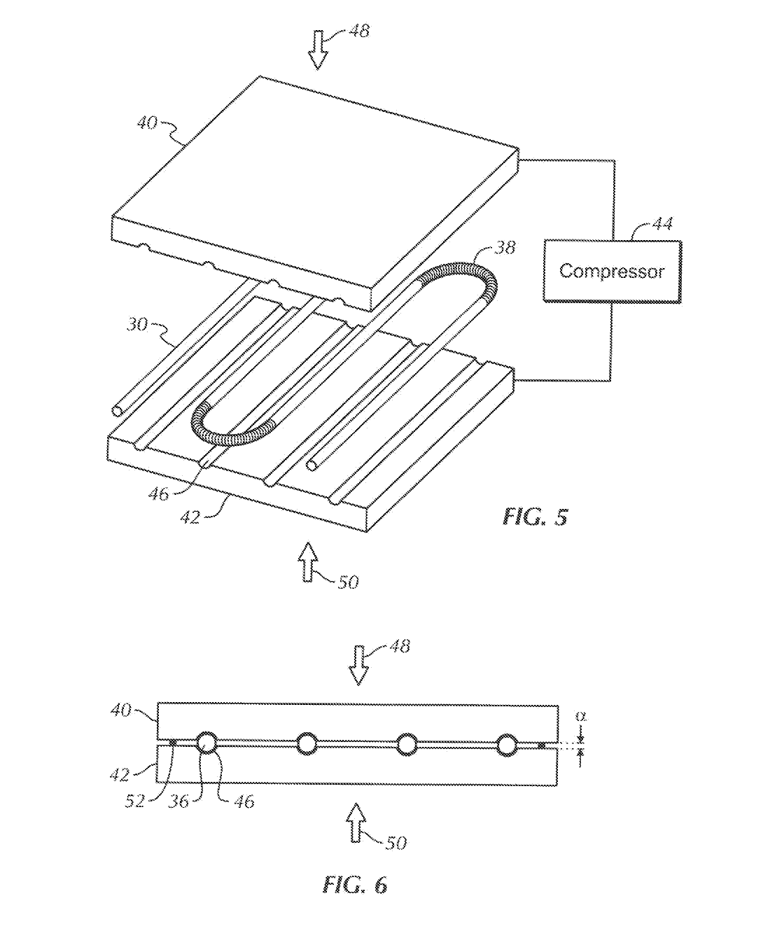

As shown in FIGS. 4-6, each HEX plate 40, 42, or at Least one plate, may be formed with straight co-parallel grooves 46 each with a transverse shape preferably configured to match the transverse cross-section of the straight segments 36 of the heat exchange member 30. In this way, physical contact between the HEX plates and straight segments is maximized. The end segments 38 of the heat exchange member 30 may be received in matching curved end segments of the grooves 46 or, to ease manufacturing, may protrude beyond the edges of the HEX plates as best shown in FIG. 5. Although in this latter configuration Less heat exchange occurs between the end segments 38 and the HEX plates 40, 42, compared to the heat exchange that occurs between the straight segments and the plates, the loss of overall neat exchange between the heat exchange member 30 and HEX plates is negligible owing to the relative short fluid path of each end segment 38, while manufacturing of the HEX plates 40, 42 may be simplified owing to grooves 46 that are straight throughout their length on the HEX plates without any curved end segments of the grooves being implemented. Optionally, the grooves may include carved portions or curved end segments. In certain embodiments, one or more heat exchange (HEX) plates may be formed with serpentine, winding, tortuous, or sinuous grooves or grooves with one or more curves, turns and/or bends, which have a transverse shape preferably configured to match the transverse cross-section of a similarly shaped heat exchange member.

One HEX plate 40, 42 may be translationally movable relative to the other end plate 42, 40 in the dimension shown by the arrows 48, 50 in FIGS. 5 and 6. Typically, this movability may be implemented by holding one of the HEX plates in a fixed location and allowing the other HEX plate to move hack and forth on, e.g., rails, although both HEX plates may be movably mounted if desired. In any case, moving the HEX plates apart facilitates insertion of the heat exchange member 30 between the HEX plates. Then, the HEX plates can be moved together and if desired clamped such that at least part of each straight segment 36 of the heat exchange member 30 is received in a corresponding groove 46 of one HEX plate 40 and/or at least part of the same straight segment 36 of the heat exchange member 30 is received in a corresponding groove 46 of the opposing HEX plate 42, as best shown in FIG. 6. In this way, at least a portion or a substantial portion of the each straight segment 36 may he in physical contact with both HEX plates 40, 42. To avoid crushing the heat exchange member 30, as shown in FIG. 6 a small gap a may exist between the HEX plates 40, 42 in the fully clamped or closed configuration shown. The gap may be, e.g., forty mils and may be imposed by one or more protrusions or beads 52 formed on one or both of the HEX plates 40, 42. While the portion of the each straight segment 36 of the heat exchange member 30 in the gap a does not contact a HEX plate 40, 42, any reduction in heat exchange caused thereby, small in any event, is balanced by the prevention of unintentional crashing of fee heat exchange member 30 between the HEX plates.

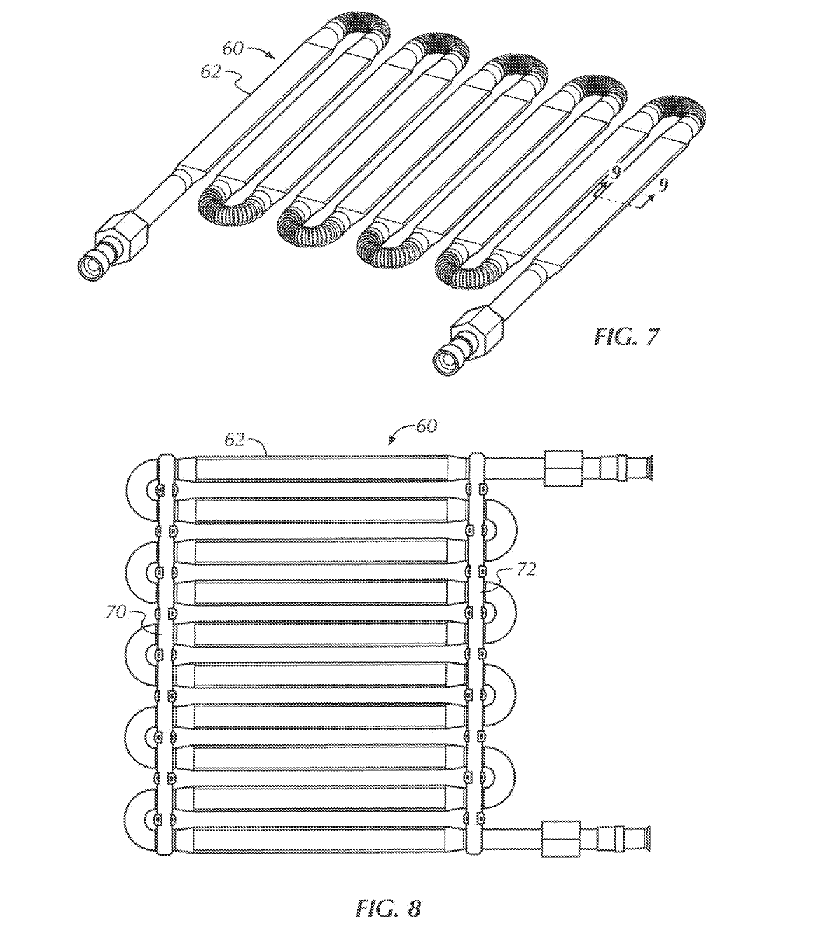

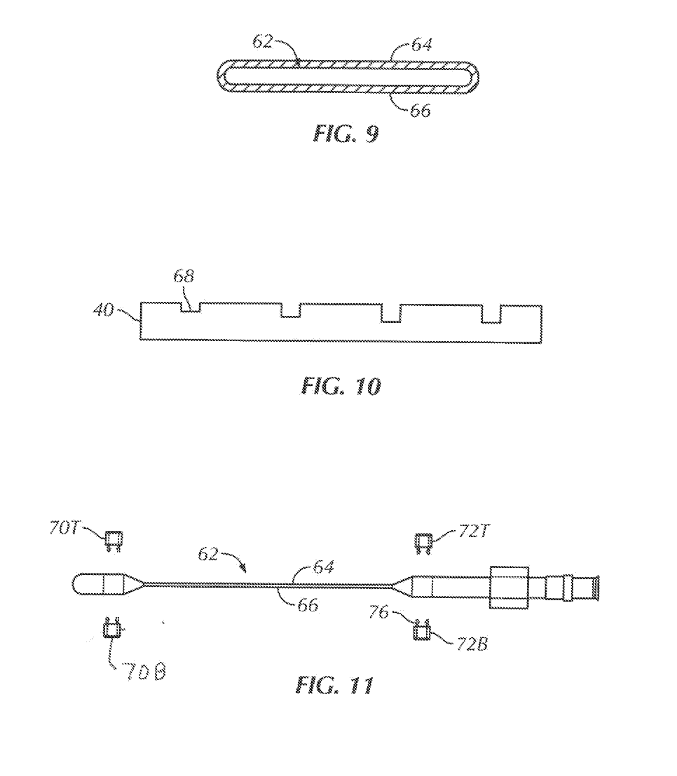

FIGS. 7-10 show an alternate heat exchange member 60 that in all essential respects is identical to the heat exchange member 30 shown in FIGS. 2-6, except that the straight segments 62 of the heat exchange member 60 may be flattened as shown, e.g., by pressing during manufacturing, such that the transverse cross-section of each straight segment 62 is non-circular as shown in FIG. 9. The heat exchange member 60 may have a serpentine, winding, tortuous, or sinuous configuration, or it may have one or more curves, turns and/or bends. It will he appreciated that the flattening of the straight segments 62 results in a cross-sectional shape that is oblong, i.e., means generally longer in the x-dimension than wide in the y-dimension. Depending on the degree of flattening, the cross-sectional shape may be elliptical, or ovular, or as shown in FIG. 9, racetrack-shaped, with top and bottom walls 64, 66 that are substantially planar, i.e., flat. In such an embodiment, each HEX plate 40, 42, or at least one plate, is formed with grooves 68 that have rectilinear cross-sectional shapes as shown in FIG. 10 to match and closely conform to the straight segments 62 of the heat exchange member 60. Such a configuration facilitates greater heat transfer by placing the working fluid flowing along the central axis of the straight segments 62 in closer proximity to the HEX plates. Moreover, grooves with rectilinear cross-sections can be easier to manufacture or form into the HEX plates than grooves with semi-circular cross-sections. In certain embodiments, as discussed supra, one or more heat exchange (HEX) plates may be formed with serpentine, winding, tortuous, or sinuous grooves or grooves with one or more curves, turns and/or bends, which have a transverse or cross-sectional shape preferably configured to match the transverse cross-section of a similarly shaped heat exchange member. FIGS. 8 and 11 best show that in some embodiments, if desired a heat exchange member as described herein, e.g., a serpentine, winding, tortuous, or sinuous heat exchange member, or heat exchange member having one or more curves, turns and/or bends, may be engaged with a frame such as a left and right pair of elongated spacer combs 70, 72. Each spacer comb 70, 72 may have a respective elongated top segment 70T, 72T (FIG. 11) and a respective elongated bottom segment 70B, 72B that can be engaged with the top segment to clamp the heat exchange member 30 or 60 there between. In the example shown, the spacer combs 70, 72 are engaged with the end segments of the heat exchange member, outboard of the straight segments, and may be formed with spacer legs 76 (FIG. 11) to maintain the spacing between adjacent sections of the heat exchange member. In certain examples, but without limitation, spacer combs may be made of injection-molded plastic. In other embodiments, the spacer combs 70, 72 maybe engaged with the straight segments of the heat exchange member, inboard of the end segments, or alternatively, a first spacer comb may be engaged with the straight segment and a second spacer comb may be engaged with the end segments. The HEX plates may be configured to accommodate one or more spacer combs, or the HEX plates may include one or more frames to maintain the spacing between adjacent sections of the heal exchange member.

The heat exchange members or assemblies and HEX plates herein maybe made of various materials, e.g., metal or thermally conductive thermoplastic or other thermally conductive materials. The heat exchange members or assemblies described herein may be disposable. In the various embodiments described herein, the straight segments may be straight, substantially straight or rectilinear. Optionally, the heat exchange members or straight segments may include one or more portions that are curved, bent, or not straight if desired. In other embodiments, a heat exchange member or assembly may be substantially straight, having none or minimal turns and/or a flattened transverse cross-section.

Components included in one embodiment can be used in other embodiments in any appropriate combination. For example, any of the various components described herein and/or depicted in the Figures may be combined, interchanged or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

While various embodiments of SERPENTINE HEAT EXCHANGE ASSEMBLY FOR REMOVABLE ENGAGEMENT WITH PATIENT HEAT EXCHANGE SYSTEM are herein shown and described in detail, the scope of the present invention is to he limited by nothing other than the appended claims.

What is claimed is:

1. A heat exchange member comprising:
a hollow body through which working fluid from an intravascular heat exchange catheter or an external heat exchange pad can flow, the body being configured for removable engagement between two plates of a heat exchange system for exchanging heat between the heat exchange system and the working fluid, the body comprising:
a working fluid inlet;
a working fluid outlet; and
a plurality of straight segments between the inlet and outlet, wherein adjacent straight segments are connected by a respective curved end segment, and at least some of the end segments are corrugated or flexible.

2. The heat exchange member of claim 1, wherein the heat exchange member includes a serpentine body.

3. The heat exchange member of claim 1, wherein the heat exchange member is made of metal or thermally conductive thermo-plastic.

4. The heat exchange member of claim 1, wherein at least one of the straight segments has a flattened transverse cross-section.

5. The heat exchange member of claim 1, wherein at least one of the straight segments defines a non-circular transverse cross-section.

6. The heat exchange member of claim 5, wherein the non-circular transverse cross-sections are oblong.

7. The heat exchange member of claim 5, wherein the non-circular transverse cross-sections have opposed flat sides.

8. The heat exchange member of claim 1, comprising the plates, the plates configured for circulating refrigerant to and from a compressor.

9. The heat exchange member of claim 8, comprising the compressor.

10. The heat exchange member of claim 1, comprising the catheter.

11. The heat exchange member of claim 1, comprising the pad.

12. A heat exchange assembly removably engageable between two heat exchange plates of a heat exchange system for exchanging heat with working fluid from an intravascular heat exchange catheter or an external heat exchange pad, the heat exchange assembly having a plurality of bends or turns and a flattened transverse cross-section to facilitate heat exchange with the heat exchange plates.

13. The assembly of claim 12, wherein at least some segments of the assembly define respective non-circular transverse cross-sections.

14. The assembly of claim 13, wherein the non-circular transverse cross-sections are oblong.

15. The assembly of claim 13, wherein the non-circular transverse cross-sections have opposed flat sides.

16. The assembly of claim 12, comprising the plates, the plates configured for circulating refrigerant to and from a compressor.

17. The assembly of claim 16, comprising the compressor.

18. The assembly of claim 12, comprising the catheter.

19. The assembly of claim 12, comprising the pad.

20. A heat exchange system comprising:
first and second heat exchange plates;
a heat exchange tubing assembly defining at least one fluid flow pathway with at least one flat segment in the fluid flow pathway and being configured for being received between the heat exchange plates to exchange heat therewith.

21. The system of claim 20, wherein the heat exchange tubing assembly includes a serpentine body or body having various turns or bends, which is supported on a frame.

22. The system of claim 20, wherein the heat exchange tubing assembly defines one and only one fluid flow pathway.
